(19) United States
(12) Patent Application Publication
(10) Pub. No.: US 2008/0090995 A1
(43) Pub. Date: Apr. 17, 2008

(54) Liquid Chromatography Method

(75) Inventors: Karin V. Andersson, Uppsala (SE); Ann Bergh, Uppsala (SE); Thomas Pless, Uppsala (SE); Jozsef Vasi, Uppsala (SE)

Correspondence Address: GE HEALTHCARE BIO-SCIENCES CORP.;PATENT DEPARTMENT, 800 CENTENNIAL AVENUE, PISCATAWAY, NJ, 08855, US

(73) Assignee: GE HEALTHCARE BIO-SCIENCES AB, UPPSALA (SE)

(21) Appl. No.: 11/814,696

(22) PCT Filed: Dec. 20, 2005
(86) PCT No.: PCT/SE05/01979

§ 371 Date: Jul. 25, 2007

(30) Foreign Application Priority Data
Feb. 14, 2005 (SE) 0500352-0

(51) Int. Cl. C07K 1/22

(52) U.S. Cl. 530/417

(57) ABSTRACT

The present invention relates to a method for the non-magnetic purification of cellular components from a crude cell lysate by continuous liquid chromatography, which method comprises lysis of cells in a vessel to provide a crude cell lysate; passing the crude cell lysate so obtained, without any intermediate clarification, over a chromatogra-phy column packed with a porous particulate chromatography matrix to adsorb target component(s), wherein the particle surfaces present immobilised nitrilotriacetic acid (NTA) ligands charged with metal ions; and recovering the target component(s) by elu-tion. The invention also encompasses a chromatography column suitable for use in the method according to the invention, which column is packed with porous non-magnetic particles having a particle size distribution of 45-165 μm, wherein the inlet and outlet means of the column are provided with deep filter units having a pore size of 20-130 μm

### TECHNICAL FIELD

The present invention relates to a method for purification of one or more cellular components such as proteins and/or peptides using continuous liquid chromatography. The invention also encompasses a chromatography column suitable for use in the method according to the invention, and a kit for purification of one or more cellular components.

### BACKGROUND

Biotechnological methods are used to an increasing extent in the production of proteins, peptides, nucleic acids and other biological compounds, for research purposes as well as in order to prepare novel kinds of drugs. Due to its versatility and sensitivity to the compounds, chromatography is often the preferred purification method in this context. The term chromatography embraces a family of closely related separation methods, which are all based on the principle that two mutually immiscible phases are brought into contact. More specifically, the target compound is introduced into a mobile phase, which is contacted with a stationary phase. The target compound will then undergo a series of interactions between the stationary and mobile phases as it is being carried through the system by the mobile phase also known as the chromatography matrix. The interactions exploit differences in the physical or chemical properties of the components in the sample.

Chromatographic methods can be run in different modes of operation. The simplest mode is batch chromatography, wherein the mobile phase is added to a vessel containing stationary phase; interaction between target compound and stationary phase is allowed for a suitable period of time; the mobile phase is withdrawn; and an eluent is added to release the target compound. In gravity chromatography on the other hand, a relatively small amount of mobile phase is added to the top of a column containing stationary phase. By opening up an outlet at the lower end of the column, the gravity will pass the mobile phase through the column, during which passage it interacts with the stationary phase. Elution is commonly performed by applying a small amount of eluent at the top, and again allowing gravity to pass it through the column. Due to the mode of operation, there is no need for distribution means at the ends of a gravity chromatography column, simple filters will do. In fluidised bed chromatography, also known as expanded bed adsorption (EBA), a liquid flow is pumped into a column containing stationary phase at the bottom, whereby the stationary phase is brought to a fluidised state, and liquid is withdrawn at the top. To improve the flow properties, the stationary phase comprises relatively heavy beads, commonly made from a polymeric material but comprising a dense core such as steel. The column used in EBA is not packed with stationary phase. Finally, in continuous liquid chromatography, a substantially constant flow of mobile phase is applied to the top of a column comprising stationary phase. By pumping the liquid through the column, a continuous flow, of controlled flow rate, is maintained during the adsorption phase. Once a suitable load of target compound has been obtained on the stationary phase, the liquid flow is changed from mobile phase to an eluent, optionally with one or more intermediate washings, and the target fraction is recovered from the eluent at the outlet of the column. The eluent will commonly comprise a gradient, such as a salt or pH gradient. To avoid contamination of large contaminants, and to obtain an advantageous liquid distribution throughout the column, the inlet is usually equipped with a filter and mechanical liquid distributor means. Most commonly, the outlet will similarly present both a filter and some mechanical liquid distributor means.

In a chromatographic purification method denoted immobilised metal ion adsorption chromatography (IMAC), interactions between a target compound and metal chelating groups present on the stationary phase are utilised. IMAC, which is also known as metal chelating affinity chromatography (MCAC), is often used for the purification of proteins. The principle behind IMAC lies in the fact that many transition metal ions can coordinate to S and N-containing groups, as are e.g. present in the amino acids histidine, cysteine, and tryptophan, via electron donor groups on the amino acid side chains. To utilise this interaction for chromatographic purposes, the metal ion must be immobilised onto an insoluble support. This can be done by attaching a chelating group to support. Most importantly, to be useful, the metal of choice must have a higher affinity for the matrix than for the compounds to be purified. Examples of suitable coordinating ions are Cu(II), Zn(II), Ni(II), Ca(II), Co(II), Mg(II), Fe(III), Al(III), Ga(III), Sc(III) etc. Various chelating groups are known for use in IMAC, such as iminodiacetic acid (IDA), which is a tridentate chelator, and nitrilotriacetic acid (NTA), which is a tetradentate chelator. The chelating groups are commonly known as ligands, while the insoluble support is known as a carrier or base matrix.

In recent years, IMAC has successfully been used for the purification of recombinant proteins and peptides, wherein histidine (His) tags have been introduced to facilitate isolation and purification. When IMAC is used for purification of recombinant proteins, in the most common process cells are lysed in a first step to free the proteins, followed by centrifugation and filtration in order to remove cell debris and other residues that would entail clogging of filters and/or inefficient adsorption to ligands. The filtered sample is subsequently combined with a binding buffer and added to the IMAC column.

U.S. Pat. No. 5,047,513 (Döbeli) relates to metal chelate resins suitable for the purification of proteins, especially those which contain neighbouring histidine residues. The disclosed purification of proteins is accomplished by subjecting the proteins to affinity chromatography on a metal chelate resin defined by the following formula:
Carrier matrix-spacer-NH—(CH₂)ₓ—CH(COOH)—N(CH₂COO⁻)₂Ni²⁺
Thus, the metal chelating affinity ligand is a nitrilotriacetic acid derivative, which is prepared by reacting an N-terminal protected compound of the formula R—HN—(CH₂)ₓ—CH(NH₂)—COOH, wherein R signifies an amino protecting group and x signifies 2, 3 or 4, with bromoacetic acid in an alkaline medium and subsequently cleaving off the protecting group.

A commercially available high throughput product that utilise the Döbeli metal chelate resins is available from Qiagen, who markets Ni—NTA magnetic agarose beads for high throughput, micro-scale purification of histidine-tagged proteins and versatile magneto-capture assays using histidine-tags. In magnetocapture, a magnet is used to retain particles in the wells as the supernatant is removed. Thus, unlike gravimetric chromatography methods, there is no need for sedimentation in magnetocapture, and unlike continuous chromatography methods, there is no flow that passes through the column. The product is available in single tubes or in 96-well microplates, and effective screening is stated to be obtainable even with crude cell lysates. An advantage of the product is that it can be used in very small volumes—as little as 10 μl can be used to purify 3 μg protein, which is convenient for high throughput micro-scale purification. The particles are 50 μm in average, but range from 20-70 μm.

Another IMAC commercially available product is marketed by BD Biosciences Clontech as the BD TALON™ CellThru Resin, which is charged with cobalt instead of nickel ions. BD TALON™ CellThru Resin is promoted for purifying proteins from non-clarified cell lysates, sonicates or fermentation liquids in expanded bed chromatography. BD TALON™ CellThru Resin comprises large agarose beads, in the range of 300-500 μm, in standard chromatography columns whose end-plate frits have 190 μm pores. According to the product note, because of the large bead size, cellular debris flows through the column between the beads while the soluble product binds to the immobilised ions on BD TALON™ CellThru Resin. As indicated above, the ligands used in this system are tetradentates based on aspartate, which are charged with cobalt (Co²⁺). As is well known, even though nickel and cobalt are both transition elements, they belong to different subgroups of the periodic table, and the binding of histidine-tagged proteins to a nickel-charged resin is as a consequence in general stronger than its binding to a cobalt-charged resin. Consequently, less stringent elution conditions may be used with a cobalt-charged resin. However, in cases when a high binding capacity is desired the stronger binding would be preferred.

WO 2004/099384 (Kappel) relates to solid phase cell lysis process and a capture platform, which more specifically comprises a mouth, an interior surface, and a coating of a lytic reagent on at least a portion of the interior surface. The amount of the lytic reagent in the coating is sufficient for the formation of a lysis solution having the capacity to lyse the host cell when a liquid suspension containing the host cell is introduced into the container. The ligand is positioned on the bottom and/or on a sidewall of the container, or on an additional support such as a bead or mesh. A variety of purification techniques are stated to be useful in this container, exemplified as metal chelate chromatography; immunogenic capture systems; glutathione-S-transferase (GST) capture and biotin-avidin/streptavidin capture systems. A stated advantage is that the disclosed system eliminates the need to centrifuge a cellular solution to remove insoluble material, pipette in additional detergent lysis liquids or enzymatic inhibitors or perform subsequent purification steps. An object of WO 2004/099384 is to provide a process which is especially advantageous in high throughput applications. The process of WO 2004/099384 represents batch-wise chromatography. As is well known, batch-wise chromatography will put fewer requirements of the equipment as regards e.g. risk of filter clogging and the like that relatively frequently appear in continuous chromatography, where the sample is brought to pass a chromatography matrix. However, continuous chromatography is often preferred for large scale operation since it reduces operation time and increases capacity.

Finally, Wlad et al (Hanna Wlad, Andras Ballagi, Lamine Bouakaz, Zhenyu Gu and Jan-Christer Janson: “Rapid Two-Step Purification of a Recombinant Mouse Fab Fragment Expressed in Escherichia coli” in Protein Expression and Purification 22, 325-329 (2001) report a large-scale process for the purification of a recombinant Fab fragment specific for tobacco mosaic virus coat protein (Fab57P). The recombinant Fab fragment was purified by two disruption of bacteria using an APV Gaulin homogenizer; cation exchange chromatography of the crude E. coli homogenate directly, without centrifugation, on a column packed with SP Sepharose™ Big Beads; and further purification by affinity adsorption to a column packed with Sepharose 6B to which an antigen peptide had been coupled.

However, despite the above-discussed methods, there is still a need in this field of alternative methods for protein and/or peptide purification, which methods can handle larger volumes of sample and provide improved binding capacities.

### BRIEF DESCRIPTION OF THE PRESENT INVENTION

A first aspect of the invention is to provide a novel method of isolating cellular components such as proteins and/or peptides from a crude cell lysate. The method is most commonly used to obtain a purified desired component, but it may also be used to remove one or more cellular components from a desired liquid.

Another aspect of the invention is to provide a liquid chromatography method for separating cellular components such as capture of proteins and/or peptides from a crude cell lysate, which provides a higher purity than the prior art methods. This can be achieved by the method defined in the appended claims, which provides improved separation of cellular components.

A specific aspect of the invention is to provide such a method, which is suitable for large scale operation i.e. preparative purification. This can be achieved by the method defined in the appended claims, which provides an improved binding capacity.

A second aspect of the present invention is to provide a packed chromatography column suitable for purification of cellular components such as proteins and/or peptides from crude cell lysates.

A specific aspect of the invention is to provide a chromatography column as described above, which allows purification of cellular components such as proteins and/or peptides from a crude cell lysate without developing a too high back pressure.

Further aspects and advantages of the present invention will appear from the description and claims below.

### BRIEF DESCRIPTION OF DRAWINGS

FIG. 1 shows protein purification by step-wise elution performed in a 1 ml column tube volume, as described in Example 1.

FIG. 2A shows protein purification by step-wise elution performed in a 5 ml column tube volume, as described in Example 2.

FIG. 2B shows the SDS-PAGE analysis performed as explained in Examples 1 and 2. The main bands in the eluted pool are GFP-His monomer and GFP-His dimer.

FIG. 3A shows protein purification by gradient elution performed as explained in Example 3. As appears from the chromatogram, the target protein elutes at 50-65 ml. FIG. 3B shows the SDS-PAGE analysis performed as explained in Example 3. The main bands in the eluted pool are GFP-His monomer and GFP-His dimer.

### DEFINITIONS

The term “crude” means unclarified.

The term “resin” refers herein to a stationary phase and is used interchangeably with other common terms such as “matrix” or “chromatography matrix”.

The term “adsorption” is used herein for the attachment (binding) of a target component to a ligand charged with metal ion.

The term “non-magnetic purification” means that there are no magnetic interactions utilised to maintain particles in the chromatography column at any stage of the process, and that process liquids such as mobile phase and eluent pass through the column without any substantial magnetic influence. The term “non magnetic particles” refers to particles commonly made from a polymeric material, to which no magnetic components have been added.

The term “peptide” is used as embracing any peptide, such as mono-, di-, oligo- and polypeptides.

### DETAILED DESCRIPTION OF THE INVENTION

In a first aspect, the present invention relates to a method for non-magnetic purification of one or more target cellular components from a crude cell lysate by continuous liquid chromatography, which method comprises
(a) lysis of cells in a vessel to provide a crude cell lysate;
(b) passing the crude cell lysate so obtained over a chromatography column packed with a porous particulate chromatography matrix to adsorb the target component(s), wherein the particle surfaces present immobilised nitrilotriacetic acid (NTA) ligands charged with one or more metal ions selected from the group that consists of Ni²⁺ ions, Cu²⁺ ions and Zn²⁺ ions; and, optionally,
(c) recovering the target component(s) by contacting the matrix with an elution buffer that releases adsorbed component(s).

In an advantageous embodiment, the crude cell lysate is passed over the chromatography column without any intermediate clarification. Chemical and mechanical lysis of cell-containing liquids such as fermentation broths are well known in this field. The chemical lysis can be carried out with any suitable lytic reagent, such as detergent, a lytic enzyme, or a chaotrope. In an advantageous embodiment, the lytic reagent used in (a) is an enzyme. In a specific embodiment, the chemical lysis is obtained by adding lysozyme in a suitable amount and under the appropriate conditions. Mechanical lysis is also well known in this field, and commonly used methods include sonication, French press cell, homogenization, grinding, and freeze-thaw lysis. As the skilled person in this field will realise, the duration of the mechanical lysis should be adapted to be long enough to avoid clogging of filters in the downstream process, but short enough not to denature the desired target proteins and/or peptides. In one embodiment, (a) comprises both a chemical and a mechanical lysis. The mechanical lysis is then performed subsequent to the chemical lysis by any well known method such as sonication. In the best embodiment of the present method, (a) comprises addition of lysozyme followed by sonication.

In the present method, the crude cell lysate so obtained is then directly added to a chromatography column, with no intermediate clarification. Thus, an advantage of the pre-sent method is that the crude cell lysate can be applied to the chromatography column without the commonly used centrifugation, filtration and/or sedimentation. The present invention shows that it is possible to obtain equivalent protein pool volume, recovery and purity as when using an unclarified lysate. In addition to the benefit of a reduced process time in terms of overall costs, the elimination of the conventionally used centrifugation and filtration steps also involves advantages such as a reduced degradation of target protein. Even though crude cell lysates have been purified using IMAC in the prior art, the present invention is the first to show that a continuous IMAC process could be applied directly on a crude cell lysate, without utilising any magnetic separation principles to aid the isolation and without developing back pressures of magnitudes that impact the purification. This was quite unexpected, since it could have been expected that cell debris and similar materials would have clogged the filters and/or impaired the performance of the chromatography matrix.

As is well known, the chromatography matrix is commonly equilibrated with a suitable binding buffer before addition of sample. The sample, which in this case is the crude cell lysate, is preferably combined with binding buffer to obtain suitable conditions for adsorption (binding). Thus, in one embodiment, in (a), the lysate is combined with a binding buffer to provide a mobile phase of suitable pH. An illustrative binding buffer will contain urea and guanidine. The volume applied will depend of the scale of the process, but may be anywhere in the range of 100-200 ml. In one embodiment, the present method is carried out in analytical scale, and the mobile phase volume is then up to 50 ml, such as 1-50 ml, for example 1 ml or 5 ml.

As mentioned above, an advantage of the present method is that unexpectedly high protein binding capacities are obtainable. Thus, in one embodiment, the protein capacity is at least 20 mg protein/ml chromatography matrix, such as at least 30 mg protein/ml chromatography matrix, and preferably at least 40 mg/ml chromatography matrix. However, it is understood by the skilled person in this field that binding capacities are dependent on the nature of the bound component, such as the nature of a protein and/or peptide, and consequently the figures above are merely general examples.

As is well known, high binding capacities is a necessity in large scale operation in order to provide an economical process. Thus, the extraordinarily high binding capacities obtained according to the present invention renders the method suitable for preparative purification. Consequently, in one embodiment, the present method is a preparative method. Thus, in an alternative embodiment, the method is carried out in large scale, i.e. preparative processing, and the volume of the mobile phase is then commonly in the range of a couple of litres up to many thousands of litres, such as from about 20-20 000 litres, e.g. about 10 000 litres.

As mentioned above, the metal chelating affinity ligands present on the particle surfaces comprise nitrilotriacetic acid (NTA), which is a tetradentate. In one embodiment, the NTA ligands are charged with Ni²⁺ ions. NTA ligands are well known in this field, see e.g. U.S. Pat. No. 4,877,830 (Döbeli). In one embodiment, the metal chelating NTA ligands have been immobilised to the porous particles via thioether coupling, see e.g. U.S. Pat. No. 6,623,655 (Kappel). However, the present invention also encompasses embodiments where the particle surface presents other metal chelating groups such as tridentates, other tetradentates or pentadentates. Such other ligands may be immobilised to the particles using any well known chemical method, such as coupling via ether, amine or amid. The metal chelating groups can be charged with any well known chelating metal, such as the ones listed in the section Background above.

In one embodiment, the porous particles have an average particle diameter in the range of 45-400 μm, and more specifically 70-200 μm, such as 90-150 μm. However, depending on the way a particulate matrix is prepared, its size distribution may vary. thus, a commonly used way of definition is by a range, wherein a specified portion of the particle diameters are found. Thus, in one embodiment of the present method, the size distribution of the porous particles are 45-165 μm, which means that at least 80%, preferably at least 95 of the particles, are within that range. In an alternative embodiment, at least 80%, preferably at least 95 of the particles, are within the range of 10-45 μm. In yet an alternative embodiment, at least 80%, preferably at least 95 of the particles, are within the range of 165-400 μm. As the skilled person in this field will realise, the choice of particle size in the present chromatography matrix will be decided on the equipment used, and in particular on the filters of the chromatography column. However, the pre-sent method can alternatively be carried out using smaller particles as well, depending on the column used. In this context, it is understood that the term “particle surface” as used herein refer to the external surface of the particle as well as to its pore surfaces. The porous particles may be made from any organic or inorganic polymer.

Thus, in one embodiment, the polymer particles are made from a native organic polymer, such as a carbohydrate, and preferably a cross-linked carbohydrate material, such as agarose, agar, cellulose, dextran, chitosan, konjac, carrageenan, gellan, alginate etc. The particles used in the present method can easily be prepared according to standard methods, such as inverse suspension gelation (S Hjertén: Biochim Biophys Acta 79(2), 393-398 (1964). Alternatively, the particles are commercially available products, such as Sepharose™ FF (Amersham Biosciences AB, Uppsala, Sweden).

In another embodiment, the polymer particles are made from a synthetic organic polymer, preferably cross-linked synthetic polymers, such as styrene or styrene derivatives, divinylbenzene, acrylamides, acrylate esters, methacrylate esters, vinyl esters, vinyl amides etc. Such particles are easily produced according to standard methods, see e.g. “Styrene based polymer supports developed by suspension polymerization” (R Arshady: Chimica e L'Industria 70(9), 70-75 (1988)). Alternatively, a commercially available product, such as Source™ (Amersham Biosciences AB, Uppsala, Sweden) can be used in the present method.

In a further embodiment, the particles are made from an inorganic material, such as silica or a ceramic, such as hydroxyapatite.

In an advantageous embodiment, the chromatography column is provided in a single use format. In this context, the term “single use” is understood to refer to one single or a very limited number of uses, as is sometimes denoted a “disposable” column. Thus, in a specific embodiment, the packed chromatography column is provided in a sterile or substantially aseptic format. Such a sterile chromatography column is advantageously used in the medical or diagnostic industry. By applying a sterile or aseptic sample to a sterile column according to the invention, the need of subsequent sterile filtration may be avoided, which is for example especially advantageous for certain larger proteins which are difficult to subject to sterile filtration. Thus, one embodiment of the present method is a method of purification of a target cellular component, which comprises sterilization of the packed column before passing the crude cell lysate across. Alternatively, the chromatography matrix and column are sterilized separately, and the matrix added to the column under aseptic conditions.

Elution of an IMAC resin is commonly performed according to standard protocols in this field, which commonly involves addition of en elution buffer comprising imidazol, and preferably also urea and guanidine. Alternatively, elution is performed by lowering the pH. The elution buffer may be added as a continuous or stepwise pH gradient. Such gradient elution would be used at least to determine the optimal elution conditions for a given process, and once such conditions have been determined an elution buffer of the optimal pH may be added in (c).

The cells from which the crude lysate originates may be any prokaryotic or eukaryotic cell, such as bacteria, yeast etc. The target component can be any cellular component, such as a polypeptide, protein, protein fragment, DNA, RNA, other nucleotide sequence, carbohydrate, lipid, cholesterol, or kinase. In one embodiment of the present method, at least one target component is a protein, and in the best embodiment, the protein is tagged with one, two or more, preferably adjacent, histidine residues. Thus, the cells are preferably recombinant cells, expressing histidine-tagged proteins or peptides. Preparing histidine-tagged proteins is well known to the skilled person, as discussed in the section Background above. A target protein may be of a size anywhere in the range of 10000-200000 Da. In one advantageous embodiment of the present method, at least one target component is a peptide, preferably a histidine-tagged peptide.

Another aspect of the present invention is a process for purification of a target cellular 5 component comprising
(a) purification of a target component using a method as described above;
(b) washing the chromatography matrix;
(c) purification of a target component using a method as described above;
   wherein (b)-(c) are repeated up to 10 times. Such washing is commonly carried out by passing a buffer such as 500 mM imidazole over the packed column to remove residuals and loosely bound components.

In one embodiment, this aspect of the invention is a process for purification of a target cellular component comprising
(a) purification of a target component using a method as described above;
(b) stripping the chromatography matrix from metal ions;
(c) subjecting the chromatography matrix to cleaning in place (cip);
(d) recharging the chromatography matrix with metal ions; and
(e) purification of a target component using a method as described above;
   wherein (b)-(e) are repeated up to 30 times.

Stripping of the column can for example be carried out with a stripping buffer comprising sodium phosphate, NaCl and EDTA, pH 7.4.

The cip of (c) can be carried out according to any well known principles, commonly depending on the nature of the chromatography matrix. An illustrative example for a polysaccharide matrix is cleaning in place with 1 M NaOH, using a contact time of 1-2 h. The cip will remove e.g. precipitated proteins, hydrophobically bound proteins, and lipoproteins. A cip may also comprise reverse flow. As is well known in this field, the number of cip cycles a chromatography matrix should be determined depending on when the specific system shows an increase in backpressure. An illustrative process may comprise e.g. up to 300 cip steps.

For a nickel chelating matrix, the recharge is e.g. carried out by loading NiSO₄ in distilled water onto the column.

In an advantageous embodiment of the present process, especially for large scale processing, the stripping of (b) is preceded by washing followed by further purification at least once. The washing may be by any suitable buffer, such as described above. The number of washing-purification cycles between each cip will vary from case to case, but the skilled person in this field can easily decide the appropriate number, 5-10 being an illustrative example.

Another aspect of the present invention is liquid chromatography column comprised of a column tube having liquid flow inlet means and liquid flow outlet means at substantially opposite ends, wherein the column tube is packed with a chromatography matrix and wherein the column has distributor means located adjacent to said inlet and outlet means, characterised in that the chromatography matrix comprises porous non-magnetic particles having a size distribution of 45-165 μm; that the particle surfaces present immobilised ligands; and that adjacent to the outlet distributor means is a deep filter unit having a pore distribution of 20-130 μm.

The particles may be of any of the above discussed materials, as long as the particle size is within the defined range. Further, the particles may comprise any kind of ligands, such as ion exchange ligands, hydrophobic interaction chromatography (HIC) ligands, reversed phase chromatography (RPC) ligands, affinity ligands or immobilised metal affinity ligands (IMAC), or multi-modal ligands, such as bimodal cation exchangers or bimodal anion exchangers.

Thus, in one embodiment, the present column comprises porous particles having immunoglobulin-binding ligands, such as protein A. An illustrative example of a commercially available such matrix is the MabSelect™ family, such as MabSelect™ Xtra and MabSelect™ Sure (Amersham Biosciences, Uppsala, Sweden), which particles are a highly flow resistant. Thus, in a specific embodiment, the particles are made as described in U.S. Pat. No. 6,602,990 (Berg), which is hereby incorporated herein via reference. Another commercially available matrix is the Capto™ family, such as CaptoQ (Amersham Biosciences, Uppsala, Sweden). This embodiment is especially advantageously run in large scale.

Another illustrative example of a suitable chromatography matrix present in the column according to the invention is a metal chelating matrix, such as IDA or NTA. Examples of commercially available NTA matrices are Ni Sepharose™ FF (Amersham Biosciences, Uppsala, Sweden) or HisSelect™ (Sigma-Aldrich).

Thus, a specific embodiment of this aspect of the invention is a liquid chromatography column comprised of a column tube having liquid flow inlet means and liquid flow outlet means at substantially opposite ends, wherein the column tube is packed with a chromatography matrix and wherein the column has distributor means located adjacent to said inlet and outlet means, characterised in that the chromatography matrix comprises porous non-magnetic particles having a size distribution of 45-165 μm; that the particle surfaces present immobilised nitrilotriacetic acid (NTA) ligands; and that adjacent to the outlet distributor means is a deep filter unit having a pore distribution of 20-130 μm.

The NTA ligands may be charged with any metal ions such as the ones listed in the section Background above, and preferably with one or more metal ions selected from the group that consists of Ni²⁺ ions, Cu²⁺ ions and Zn²⁺ ions. In an advantageous embodiment, the NTA ligands have been charged with Ni²⁺ ions. In an advantageous embodiment, the NTA ligands have been immobilised to the porous particles via thioether coupling. Immobilisation of ligands was discussed above in the context of the first aspect of the invention.

In one embodiment, the porous particles have an average particle diameter in the range of 45-400 μm, and more specifically 70-200 μm, such as 90-150 μm. In a specific embodiment, the porous particles have an average particle diameter of about 90 μm. However, depending on the way a particulate matrix is prepared, its size distribution may vary. thus, a commonly used way of definition is by a range, wherein a specified portion of the particle diameters are found. Thus, in one embodiment of the present method, the size distribution of the porous particles are 45-165 μm, which means that at least 80%, preferably at least 95 of the particles, are within that range.

The deep filter units of the present chromatography column are commercially available, e.g. from Basell. Distribution means are also available on the market, and the skilled person can easily provide the parts that constitute the column according to the invention. The column tube may be made any suitable and well known material, such as glass or plastic materials.

In one embodiment, the present column presents an analytical scale column tube volume, as discussed in more depth above.

In one embodiment of the present column, the column tube volume is suitable for preparative purification of proteins and/or peptides, as discussed in more depth above.

The present invention also relates to a multi-well plate comprising at least two liquid chromatography columns as described above. Multi-well formats are well known in this field, and the skilled person can easily prepare such plates based on the teachings of the present invention and his general knowledge of the field.

An advantageous embodiment of the multi-well format is an automated system for protein and/or peptide purification comprising at least one multi-well plate according to the invention. The present method is especially suitable for automation, since it is a continuous method and comprise fewer process steps than the prior art. The skilled person can easily automate the present method and/or adapt it for multi-well format based on the teachings of the present invention and his general knowledge of the field.

The present invention also encompasses the use of a column according to the invention in a method as described above. Thus, details of this aspect may be found in the detailed description above. However, an advantageous example of use of the present invention is in the preparation of protein-based drugs or diagnostic agents, preferably for use within the rapidly expanding field of personalized medicine. Another use of the present invention within personalized healthcare is using the method to diagnose a patient. In this embodiment, the method is quantitatively used for identification, such as by detecting the presence of a target cellular component by binding thereof to the herein disclosed chromatography matrix directly from a crude cell lysate.

Finally, the invention also encompasses a kit comprising, in separate compartments, a column according to the invention; one or more metal ions selected from the group that consists of Ni²⁺ ions, Cu²⁺ ions and Zn²⁺ ions; and at least one buffer. In one embodiment of the kit, one buffer is a binding buffer comprising urea or guanidine. The column according to the invention, the chromatography matrix and other details may be as discussed above. Thus, in one embodiment, the kit comprises a disposable chromatography column.

The kit may also comprise additional equipment useful with the column, such as luer adaptors, tubing connectors, and domed nuts. In another embodiment, one buffer is an elution buffer comprising imidazole. In an advantageous embodiment, the kit comprises written instructions, preferably describing protein and/or peptide purification from a crude cell lysate.

### DETAILED DESCRIPTION OF DRAWINGS

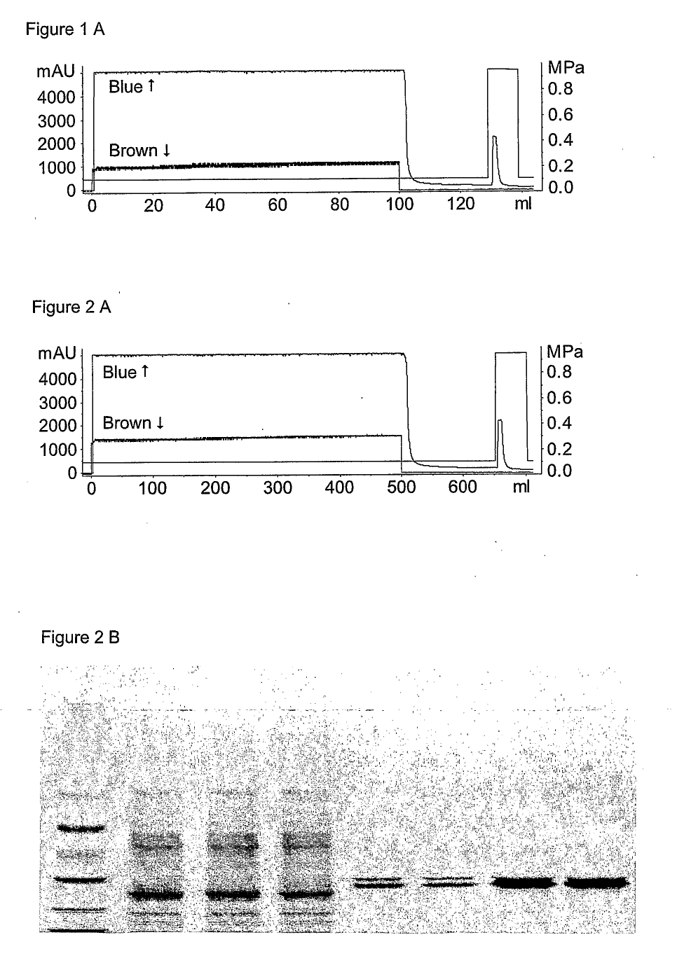

FIG. 1 shows protein purification by stepwise elution performed as described in Example 1. The imidazole concentration in binding buffer and sample is 45 mM in order to obtain a pure target protein, i.e. less binding of contaminating E. coli proteins. The target protein is eluted at approx. 130 ml (above the linear absorbance range, <2000 mAU). The blue line (upper) shows the absorbance at 280 nm, while the brown line (lower) shows the pressure during sample application.

FIG. 2A shows protein purification by step-wise elution performed as described in Example 2. The imidazole concentration in binding buffer and sample is 45 mM in order to obtain a pure target protein, i.e. less binding of contaminating E. coli proteins. The target protein is eluted at approx. 130 ml (above the linear absorbance range, <2000 mAU). The blue line (upper) shows the absorbance at 280 nm, while the brown line (lower) shows the pressure during sample application.

FIG. 2B shows the SDS-PAGE analysis performed as explained in Examples 1 and 2. In the gel shown, Band 1: LMW; Band 2: Start material; Band 3: Flow through column, Ex 1; Band 4: Flow through, Ex 2; Band 5: Wash, Ex 1; Band 6: Wash, Ex 2; Band 7: Eluted pool, Ex 1, Band 8: Eluted pool, Ex 2. The main bands in the eluted pool are GFP-His monomer and GFP-His dimer.

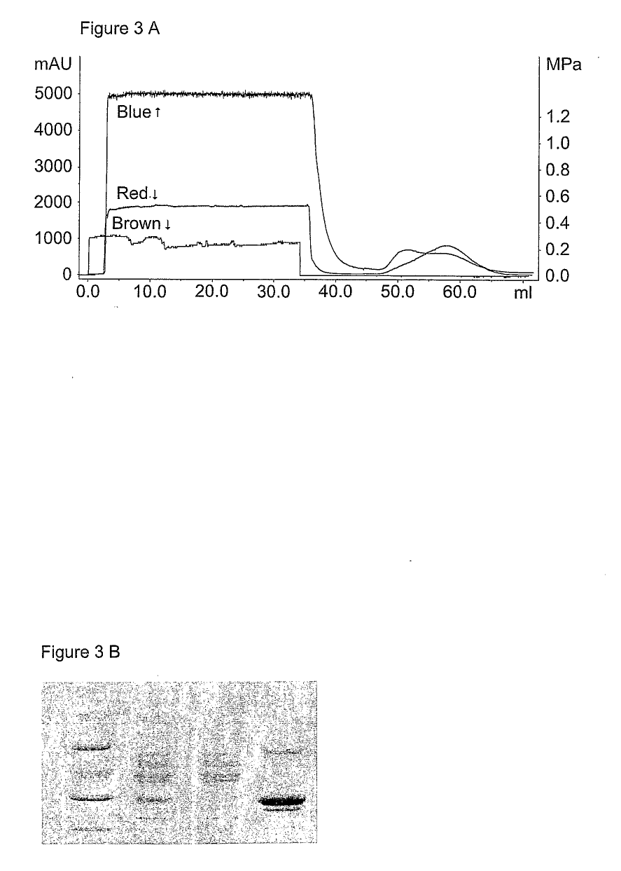

FIG. 3A shows protein purification by gradient elution performed as explained in Example 3. As appears from the chromatogram, the target protein elutes at 50-65 ml. The elution is indicated by the absorbance at 490 nm (more specific for the target protein). The preceding peak contains contaminating E. coli proteins. The blue line shows the absorbance at 280 nm; the red line the absorbance at 490 nm, and the brown line the pressure during sample application.

FIG. 3B shows the SDS-PAGE analysis performed as explained in Example 3. In the gel shown, Band 1: LMW; 2: Start material; Band 3: Flow through; and Band 4 Eluted pool. The main bands in the eluted pool are GFP-His monomer and GFP-His dimer.

Experimental Part

The present examples are provided for illustrative purposes only, and should not be construed as limiting the invention as defined by the appended claims. All references given below and elsewhere in the present specification are hereby included herein via reference.

Materials and Methods

The chromatography tube was made from PP Moplen HP 400 R (Basell); arranged with conventional means from PIAB (56110890 and 56110889); Frohe AB (56324771, 72, 73, 74); Silva Plastic Center AB (56104640 and 56102939); and MicroPlast AB (56320264). Examples 1 and 3: Height 25.2 mm, diameter 7.2 mm. Example 2: Height 25.2 mm, diameter 16.2 mm.

The deep filter unit was Vyon F, material HDPE, pore size 25-127 μm (PIAB).

The chromatography column was packed with Ni Sepharose™ HP (Amersham Biosciences, Sweden) using standard packing procedure.

### EXAMPLE 1

### Purification of Green Fluorescent Protein Using Stepwise Elution

The column volume in this experiment was 1 ml. The sample volume was 100 ml and the sample load 20 mg. The sample, was histidine-tagged green fluorescent protein (GFP-(His)₆) in unclarified E. coli BL-21 lysate.

The theoretical molecular weight, Mr, for GFP-(His)₆ is 28 197 and pI 6.1. The clone was obtained from Dr. David Drew, Stockholm University.

Fermentation (E. coli BL21 [DE3] cells) was performed according to standard methods in a medium comprising 100 μg/ml carbenicillin and 25 μg/ml chloramphenicol, glucose added, induced during 4 h by IPTG 0.8 mM, to an OD₆₀₀ of about 25. The cells were homogenised and partly purified to give a preparation with an estimated concentration of GFP-His of approximately 12 mg/ml.

The partially purified GFP-(His)₆ was spiked into (=added to) an extract from non-transformed E. coli BL21 consisting of 5 ml binding buffer per gram of cell paste. The final concentration of GFP-(His)₆ in the sample was 0.2 mg/ml.

Enzymatic lysis was performed by additions of 0.2 mg/ml lysozyme, 20 μg/ml DNAse and 1 mM MgCl₂ (final concentrations). The protease inhibitor Pefabloc™ SC was added to a final concentration of 1 mM. Lysis was performed during stirring for 30 minutes at room temperature. Finally the sample was homogenised and pH adjusted to pH 7.4.

The mobile phase comprised binding buffer: 45 mM imidazole, 0.5 M sodium chloride, 20 mM sodium phosphate pH 7.4

The elution was performed with elution buffer: 500 mM imidazole, 0.5 M sodium chloride, 20 mM sodium phosphate, pH 7.4

Chromatographic system: ÄKTA™ Explorer 10 (Amersham Biosciences, Sweden) Flow rate: 1 ml/min.

Chromatographic method:
Equilibration: 5 column volumes (CV) binding buffer
Sample application: 100 ml sample
Wash out unbound proteins: 20 CV binding buffer
Elution: 10 CV elution buffer

The resulting chromatogram is shown in FIG. 1.

Non-reduced SDS-PAGE was used to analyse the peak from the chromatogram and was performed according to Instructions for ExcelGel SDS (#80-1310-00). A gradient gel 8-18% was used. The sample was mixed 1:1 with 2× sample buffer (non-reduced) and heated for 5 minutes at 95° C. 20 μl of the sample cocktail was applied to the paper pieces on the gel. The limiting settings on the power supply were: 600 V, 50 mA, 30 W. The gel was Coomassie-stained.

The resulting gel is shown in FIG. 2B, together with the analysis of the peak obtained according to Example 1.

### EXAMPLE 2

### Purification of Green Fluorescent Protein Using Stepwise Elution

The column volume in this experiment was 5 ml. The sample volume was 500 ml and the sample load 100 mg. The sample, was histidine-tagged green fluorescent protein (GFP-(His)₆) in unclarified E. coli BL-21 lysate.

The theoretical molecular weight, Mr, for GFP-(His)₆ is 28 197 and pI 6.1. The clone was obtained from Dr. David Drew, Stockholm University.

Fermentation (E. coli BL21 [DE3] cells) was performed according to standard methods in a medium comprising 100 μg/ml carbenicillin and 25 μg/ml chloramphenicol, glucose added, induced during 4 h by IPTG 0.8 mM, to an OD₆₀₀ of about 25. The cells were homogenised and partly purified to give a preparation with an estimated concentration of GFP-His of approximately 12 mg/ml.

The partially purified GFP-(His)₆ was spiked into (=added to) an extract from non-transformed E. coli BL21 consisting of 5 ml binding buffer per gram of cell paste.

The final concentration of GFP-(His)₆ in the sample was 0.2 mg/ml.

Enzymatic lysis was performed by additions of 0.2 mg/ml lysozyme, 20 μg/ml DNAse and 1 mM MgCl₂ (final concentrations). The protease inhibitor Pefabloc™ SC was added to a final concentration of 1 mM. Lysis was performed during stirring for 30 minutes at room temperature. Finally the sample was homogenised and pH adjusted to pH 7.4.

The mobile phase comprised binding buffer: 45 mM imidazole, 0.5 M sodium chloride, 20 mM sodium phosphate pH 7.4

The elution was performed with elution buffer: 500 mM imidazole, 0.5 M sodium chloride, 20 mM sodium phosphate, pH 7.4

Chromatographic system: ÄKTA™ Explorer 10 (Amersham Biosciences, Sweden) Flow rate: 5 ml/min.

Chromatographic method:
Equilibration: 5 column volumes (CV) binding buffer
Sample application: 100 ml sample
Wash out unbound proteins: 20 CV binding buffer
Elution: 10 CV elution buffer

The resulting chromatogram is shown in FIG. 2A.

Non-reduced SDS-PAGE was used to analyse the peak from the chromatogram and was performed according to Instructions for ExcelGel SDS (#80-1310-00). A gradient gel 8-18% was used. The sample was mixed 1:1 with 2× sample buffer (non-reduced) and heated for 5 minutes at 95° C. 20 μl of the sample cocktail was applied to the paper pieces on the gel. The limiting settings on the power supply were: 600 V, 50 mA, 30 W. The gel was Coomassie-stained.

The resulting gel is shown in FIG. 2B, together with the analysis of the chromatographic peak obtained according to Example 1.

### EXAMPLE 3

### Gradient Elution

The column volume in this experiment was 1 ml. The sample volume was 36 ml and the sample load 36 mg. The sample, was histidine-tagged green fluorescent protein (GFP-(His)₆) in unclarified E. coli BL-21 lysate.

The theoretical molecular weight, Mr, for GFP-(His)₆ is 28 197 and pI 6.1. The clone was obtained from Dr. David Drew, Stockholm University. Fermentation (E. coli BL21 [DE3] cells) was performed according to standard methods in a medium comprising 100 μg/ml carbenicillin and 25 μg/ml chloramphenicol, glucose added, induced during 4 h by IPTG 0.8 mM, to an OD₆₀₀ of about 25. The cells were homogenised and partly purified to give a preparation with an estimated concentration of GFP-His of approximately 12 mg/ml. The partially purified GFP-(His)₆ was spiked into (=added to) an extract from non-transformed E. coli BL21 consisting of 5 ml binding buffer per gram of cell paste. The final concentration of GFP-(His)₆ in the sample was 1.0 mg/ml. Enzymatic lysis was performed by additions of 0.2 mg/ml lysozyme, 20 μg/ml DNAse and 1 mM MgCl₂ (final concentrations). The protease inhibitor Pefabloc™ SC was added to a final concentration of 1 mM. Lysis was performed during stirring for 30 minutes at room temperature. Finally the sample was sonicated for 7 minutes and pH was adjusted to pH 7.4.

The mobile phase comprised binding buffer: 5 mM imidazole, 0.5 M sodium chloride, 20 mM sodium phosphate pH 7.4

The elution was performed with elution buffer: 500 mM imidazole, 0.5 M sodium chloride, 20 mM sodium phosphate, pH 7.4

Flow rate: 1 ml/min.

Chromatographic system: ÄKTA™ Explorer 100 (Amersham Biosciences, Sweden)

Chromatographic method:
Equilibration: 5 column volumes (CV) binding buffer
Sample application: 36 ml sample
Wash out unbound proteins: 10 CV binding buffer
Gradient elution: 20 CV 1-50% elution buffer (5-250 mM imidazole)

The resulting chromatogram is shown in FIGS. 3A and B.

Non-reduced SDS-PAGE was used to analyse the peak from the chromatogram obtained as described above. The SDS-PAGE analysis was performed according to Instructions for ExcelGel SDS (#80-1310-00). A gradient gel 8-18% was used. The sample was mixed 1:1 with 2× sample buffer (non-reduced) and heated for 5 minutes at 95° C. 20 μl of the sample cocktail was applied to the paper pieces on the gel. The limiting settings on the power supply were: 600 V, 50 mA, 30 W. The gel was Coomassie-stained.

1: A method for non-magnetic purification of one or more target cellular components from a crude cell lysate by continuous liquid chromatography, which method comprises:
(a) lysis of cells in a vessel to provide a crude cell lysate;
(b) passing the crude cell lysate so obtained over a chromatography column packed with a porous particulate chromatography matrix to adsorb the target component(s), wherein the particle surfaces present immobilised nitrilotriacetic acid (NTA) ligands charged with metal ions selected from the group consisting of Ni²⁺ ions, Cu²⁺ ions and Zn²⁺ ions; and, optionally,
(c) recovering the target component(s) by contacting the matrix with an elution buffer that releases adsorbed component(s).

2: The method of claim 1, wherein the lysis of (a) comprises chemical lysis.

3: The method of claim 1, wherein the lysis of (a) comprises mechanical lysis.

4: The method of claim 1, wherein the lysis of (a) comprises chemical lysis followed by mechanical lysis.

5: The method of claim 1, wherein a target component is a protein and/or peptide.

6: The method of claim 5, wherein the protein and/or peptide is tagged with at least one histidine residue.

7: The method of claim 1, wherein the binding capacity of the chromatography matrix is at least about 30 mg protein/ml matrix, preferably at least about 40 mg protein/ml chromatography matrix.

8: The method of claim 1, which is carried out using volumes and flow rates of preparative scale.

9: The method of claim 1, which is carried out using volumes and flow rates of analytical scale.

10: The method of claim 1, wherein the NTA ligands have been immobilised to the porous particles via thioether coupling.

11: The method of claim 1, wherein the NTA ligands are charged Ni²⁺ ions.

12: The method of claim 1, wherein the size distribution of the porous particles are 45-165 μm.

13: A process for purification of a target cellular component comprising:
(a) purification of a target component using the method of claim 1;
(b) washing the chromatography matrix;
(c) purification of a target component using the method of claim 1;
wherein (b)-(c) are repeated up to 10 times.

14: A process for purification of a target cellular component comprising:
(a) purification of a target component using the method of claim 1;
(b) stripping the chromatography matrix from Ni²⁺ ions;
(c) subjecting the chromatography matrix to cleaning in place (cip);
(d) recharging the chromatography matrix with Ni²⁺ ions; and
(e) purification of a target component using the method of claim 1;
wherein (b)-(e) are repeated up to 30 times.

15: The process of claim 14, wherein the stripping of (b) is preceded by washing followed by further purification at least once.
